(19) United States
(12) Patent Application Publication
(10) Pub. No.: US 2014/0081359 A1
(43) Pub. Date: Mar. 20, 2014

(54) ENHANCED LIGHT BASED LIPOPLASTY

(71) Applicant: Bruce J. SAND, Westlake Village, CA (US)

(72) Inventor: Bruce J. SAND, Westlake Village, CA (US)

(21) Appl. No.: 13/623,040

(22) Filed: Sep. 19, 2012

(51) Int. Cl. A61N 5/06

(52) U.S. Cl. CPC A61N 5/0613
USPC 607/90; 607/88

(57) ABSTRACT

A light applicator configured for contacting a portion of a subject's body surface and applying light irradiation thereto for lipolysis of target tissue underlying the subject's body surface is provided. The light applicator includes: a substrate; a plurality of light emitting diodes (LEDs) arranged on the substrate. A beam divergence of each of the plurality of LEDs is in the range of 50 to 70 steradian. The plurality of LEDs is arranged such that their emitted light beams overlap during transmission to a target tissue underlying the subject's body surface.

### TECHNICAL FIELD

The present invention relates to apparatus, compositions and methods for enhancing light based lipolysis.

### BACKGROUND

Various apparatus are known for non-invasive low-power laser irradiation of a subject's skin for lipolysis of underlying adipose tissue. Some example apparatus, systems and methods are described in commonly-assigned U.S. Pat. No. 7,959,656 and U.S. patent application Ser. No. 11/860,457, which are hereby incorporated by reference herein in their entirety. Some known apparatus include laser diodes mounted in applicators. The applicators are placed in contact with the subject's skin. Laser diodes have drawbacks including high cost, high energy requirements, high heat generation, and potential damage to the subject's eyes.

Cost-effective, efficient and safe apparatus, compositions and methods for light based lipolysis are desirable.

### SUMMARY OF THE INVENTION

Embodiments of the present invention are directed to apparatus for light emitting diode (LED) light based lipolysis of adipose tissue. Embodiments of the present invention are also directed to compositions and methods for improving penetration of light to adipose tissue to enhance light based lipolysis. Embodiments of the present invention are also directed to compositions and methods for enhancing light based lipolysis of adipose tissue by delivering lipomodulating agents to the adipose tissue. Embodiments of the present invention are also directed to compositions and methods for enhancing light based lipolysis of adipose tissue by applying skin tone restoration agents to skin overlying adipose cells subjected to lipolysis.

According to one aspect of the invention, a light applicator configured for contacting a portion of a subject's body surface and applying light irradiation thereto is provided. The light applicator includes: a substrate; a plurality of light emitting diodes (LEDs) arranged on the substrate. A beam divergence of each of the plurality of LEDs is in the range of 50 to 70 steridian. The plurality of LEDs is arranged such that their emitted light beams overlap during transmission to a target tissue underlying the subject's body surface.

The beam divergence of each of the plurality of LEDs may be about 60 steridian. Spacing between adjacent LEDs may be no greater than 1.0 cm. The substrate comprises a deformable material. The light applicator may include a cover for covering at least the plurality of LEDs and substantially transparent to light emitted by the LEDs. The cover may have a substantially flat surface for contacting a portion of the subject's body surface. The cover may be made of a deformable material. The plurality of LEDs emit may light at a wavelength ranging from about 625 nm to about 880 nm, or about 625 nm to about 680 nm.

According to another aspect of the invention, a method for inducing lipolysis in a target adipose tissue site is provided. The method includes the step of irradiating a skin surface with light to induce lipolysis at the target adipose tissue site by contacting the skin surface with a light applicator as described above. The step of contacting the skin surface may include conforming the light applicator to a contour of the skin surface.

According to another aspect of the invention, a lipolysis system is provided. The lipolysis system includes a plurality of light applicators as described above and a control device. Each of the plurality of light applicators is in communication with the control device. The plurality of light applicators may be detachably connected in series.

According to another aspect of the invention, a method for inducing lipolysis in a plurality of target adipose tissue sites is provided. The method includes the step of irradiating a plurality of skin surfaces with light to induce lipolysis at the target adipose tissue sites by contacting each of the plurality of skin surface with a light applicator of the lipolysis system described above.

According to another aspect of the invention, a method for inducing lipolysis in a target adipose tissue site is provided. The method includes the step of irradiating a skin surface with light to induce lipolysis at the target adipose tissue site by conformingly contacting the skin surface with the lipolysis system described above.

### BRIEF DESCRIPTION OF THE DRAWINGS

In the drawings which show non-limiting embodiments of the invention:

FIG. 1 is a schematic view of an LED applicator according to an embodiment of the invention;

FIG. 2 is a cross-sectional side view of the LED applicator of FIG. 1 taken along the plane A-A;

FIG. 3 is a cross-sectional side view of an LED applicator according to an embodiment of the invention;

FIG. 4 is a schematic view of an LED applicator system according to an embodiment of the invention;

FIG. 5 is a schematic view of an LED applicator system according to an embodiment of the invention;

FIGS. 6A and 6B are pre-treatment and post-treatment photographs of the thighs of a representative subject undergoing treatment with a lipomodulation composition according to an embodiment of the invention;

FIGS. 7A and 7B are pre-treatment and post-treatment photographs of the abdomen of a representative subject undergoing treatment with a lipomodulation composition according to an embodiment of the invention; and

FIGS. 8A and 8B are pre-treatment and post-treatment photographs of the face of a representative subject undergoing treatment with a skin tone restoration composition according to an embodiment of the invention.

### DETAILED DESCRIPTION OF THE PREFERRED EMBODIMENTS

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and the drawings are to be regarded in an illustrative, rather than a restrictive, sense.

Embodiments of the present invention relate to apparatus for LED light based lipolysis of adipose tissue. Embodiments of the present invention also provide compositions for application to a subject's skin overlying adipose tissue in conjunction with treatment of the adipose tissue with LED light or laser diode light; these compositions and related methods (i) enhance penetration of the light, (ii) stimulate lipolysis and/or inhibit lipogenesis, and/or (iii) restore tone to skin made flaccid by light-based lipolysis of adipose tissue. Target areas for lipolysis include the arms, thighs, buttocks, torso and chin.

### (i) LED Light-Based Lipolysis

The inventor has determined that certain characteristics of laser light such as coherence and collimation are not necessary to effect lipolysis in adipocytes. Certain aspects of the present invention relate to the use of arrays of light emitting diodes (LEDs), as a substitute for laser diodes, for light based lipolysis.

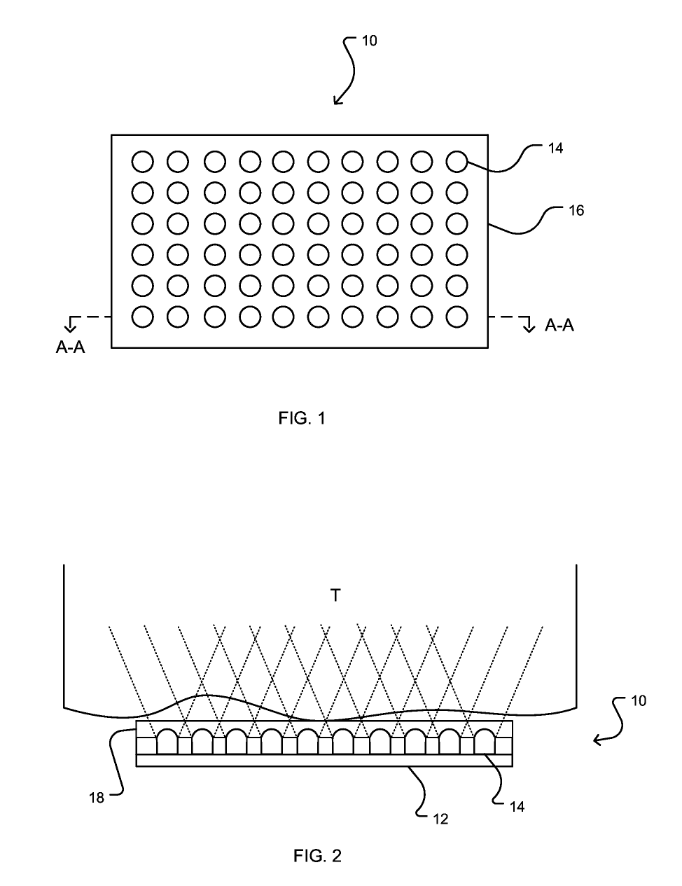

FIGS. 1 and 2 show an LED applicator 10 according to an example embodiment of the invention. Applicator 10 may be dimensioned to be manipulable by hand. The size and shape of applicator 10 may for example depend on the shape, contour and/or size of the region(s) of skin overlying the target area in the subject's body. Applicator 10 may for example be in the shape of a rectangular panel as illustrated in FIGS. 1 and 2 with dimensions of approximately 6 cm×12 cm×0.5 cm.

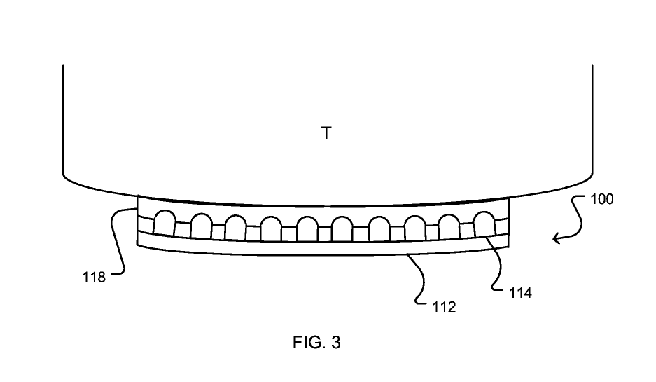

Applicator 10 includes a substrate 12. In some embodiments substrate 12 may be a rigid plate. In other embodiments such as applicator 100 shown in FIG. 3, substrate 112 may comprise a deformable material that conforms to the surface contour of the subject's body.

A plurality of LED elements 14 is arranged on substrate 12. In the illustrated embodiment substrate 12 is rectangular and sixty LED elements 14 are arranged in a six by ten matrix. In other embodiments substrate 12 may be in other shapes with different arrangements and/or different numbers of LED elements 14 arranged thereon. For example, in some embodiments the number of LED elements 14 may range from 10 to 60. The shape of substrate 12 and the arrangement and/or number of LED elements 14 may for example depend on the shape, contour and/or size of the region(s) of skin overlying the target area in the subject's body.

LED elements 14 emit light in a wavelength range suitable for penetration to adipocytes in the subcutaneous skin layer and non-thermal activation of lipolysis. In some embodiments, the wavelength range may be in the for example be about 625 nm to about 880 nm, or about 625 nm to about 680 nm. In some embodiments LED elements 14 emit light in the visible red (635 to 680 nm) to near infrared (NIR) (780 to 980 nm) wavelength ranges. In some embodiments LED elements 14 emit light at wavelengths suitable for both lipolysis and photoactivation therapy, for example wavelengths of about 633 nm and/or about 830 nm. Lipolysis may result from activation of a photochemical cascade, passive transient disruption of adipocyte membranes and/or up-regulation of enzymatic conversion of intracellular triglycerides to free fatty acids and glycerol. Photoactivation therapy may involve enhancing cell function through a photochemical cascade-mediated rise in intracellular concentrations of ATP, calcium ions, and protons, and/or activation of cytochrome C oxidase.

In some embodiments, the light emitted by LED elements 14 has a power output of about 10 mW to about 160 mW, or about 10 mW to about 100 mW. The beam divergence of LED elements 14 may range from about 50 to about 70 steridian, or about 60 steridian. LED elements 14 may emit quasi-monochromatic light. LED elements 14 may have a wavelength bandwidth of between 20 to 30 nm, or about 25 nm.

LED elements 14 dissipate less heat than laser diodes and therefore are able to be spaced closer together than laser diodes. As shown in FIG. 2, LED elements 14 are spaced close enough together that their emitted light beams overlap during transmission to a target area in tissue T. In some embodiments LED elements may be spaced no more than 1.0 cm, or no more than 0.5 cm, from each other. The overlap results in an interference effect that provides increased photon density at the target area compared to the photon density in the absence of overlap. The interference effect may include forward and backward scattering of red and NIR light. The resulting broad and intense beam of light from the plurality of LED elements 14 allows lipolysis over a correspondingly broad target area.

As shown in FIG. 2, contact surface 16 includes a cover 18. Cover 18 has a smooth surface. Cover 18 spans at least all LED elements 14. Cover 18 is substantially transparent to the light emitted by LED elements 14. Cover 18 prevents debris from collecting on the surface of LED elements 14, facilitates cleaning of contact surface 16 between treatments, and facilitates any heat dissipation by directing heat posteriorly through cover 18 that. In some embodiments, as shown in FIGS. 1 and 2, cover 18 may comprise a rigid plate. In other embodiments such as applicator 100 as shown in FIG. 3, cover 118 may comprise a deformable material that can conform to the surface contour of the subject's body.

In some embodiments, a control device (not shown) comprising hardware and circuitry for controlling and powering applicators 10, 100 may be hardwired or wirelessly connected to applicators 10, 100. In other embodiments, the control device may be incorporated into a component of applicators 10, 100, such as substrates 12, 112.

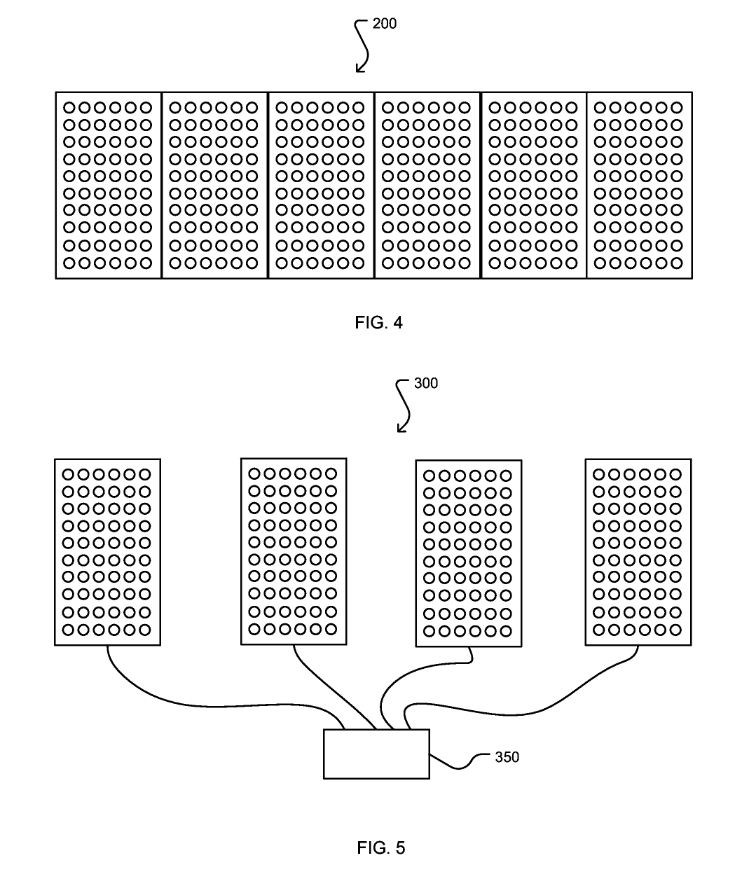

FIG. 4 shows an applicator system 200 according to an embodiment of the invention. Applicator system 200 comprises a plurality of applicators, such as applicators 10, 100, for simultaneous irradiation of large areas of a subject's body such as the waist or thigh. The applicators may be detachably joined along one or more sides, for example with hook and loop fasteners, slide fasteners, snap fasteners or the like. In the illustrated embodiment six applicators are serially joined to form a belt-like component for wrapping around a region of the subject's body. In other embodiments the applicator system may comprise more or less applicators.

FIG. 5 shows an applicator system 300 according to another embodiment. Applicator system 300 comprises a plurality of individual applicators, such as applicators 10, 100, for simultaneous irradiation of different skin surface regions of a subject's body. Applicator system 300 includes a control device 350 connected to each applicator (shown as a hardwire connection but could in alternative embodiments be a wireless connection) comprising hardware and circuitry for controlling and powering the applicators.

In operation, applicator(s) 10, 100 may be directly applied against a portion of a subject's body surface overlying a target area for fat reduction. Applicator(s) 10, 100 may be held against the skin by hand, an adhesive patch or strap, for example. LED elements 14, 114 may be operated in a continuous wave mode. The inventor has determined that the continuous wave mode induces enhanced light propagation at the target area for lipolysis.

The preselected irradiation treatment time may range from 10 to 20 minutes, for example. In some embodiments, the power supply of applicator 10 may include a timing device for adjusting the preselected irradiation treatment time to between 2 and 20 minutes, and automatically shutting off the power supply upon reaching the preselected irradiation time.

The subject may be positioned in a supine, prone or upright position during treatment. The subject may wear eye protection, such as eye cups, during treatment.

In some embodiments, a treatment regimen may comprise three treatments per week on an alternating day interval for three weeks. Treatment regimens may include pre and post treatment measurement and recording of relevant size measurements (e.g. circumference) of the relevant regions of the body to assess fat reduction.

The compositions and methods described next may for example be used in conjunction with the LED-based apparatus and methods described above, as well as low level laser diode-based apparatus, systems and methods, for example those described in commonly-assigned U.S. Pat. No. 7,959,656 and U.S. patent application Ser. No. 11/860,457. The term “low level laser” refers to laser light generated by laser diodes where the power output of an individual laser diodes does not exceed 500 mW.

### (ii) Enhanced LED or Laser Light Penetration

The inventor has determined that turbidity of skin tissue can interfere with irradiation of adipose tissue. Turbidity of skin tissue causes scattering of light before it penetrates through to the adipose tissue. Light scattering results in less light reaching the adipose tissue, hampering the therapeutic effect of irradiation (i.e., lipolysis). Reduced therapeutic efficacy prolongs a subject's irradiation exposure time required to achieve the desired therapeutic result (i.e., fat loss). Prolonged subject irradiation exposure time is undesirable for the user and the subject.

According to one embodiment of the invention, a composition comprising at least one optical skin clearing agent is provided. The optical skin clearing agent may be a hyperosmotic agent. The hyperosmotic agent may be glycerol, polyethylene glycol (PEG), polypropylene glycol (PPG), polymers thereof, combinations thereof, and the like. The hyperosmotic agent dehydrates the skin to reduce the difference in the refractive indices of skin components such as ground substance (extrafibrillar matrix) and dermal collagen. Reducing the refractive index mismatch between skin components reduces light scattering and improves penetration of LED or laser light through the skin to underlying adipose tissue. Increased light penetration, in turn, enhances lipolysis in adipose tissue.

In some embodiments, the composition may comprise lipophilic PPG-based polymers and hydrophilic PEG-based polymers. In some example embodiments, the composition comprises PPG and PEG in a 1:1 ratio.

In some embodiments the composition is topically applied to skin overlying the targeted adipose tissue about 5 minutes to about 60 minutes prior to irradiation. The composition may, for example, be applied approximately 5 to 10 minutes prior to irradiation when combined with a transepidermal delivery agent as described below. In an example embodiment, the composition may be applied onto to the skin surface at 0.2 mL per 2×2 cm area. The composition may be applied and covered with an occlusive dressing.

In some embodiments, the inventor has determined that it is advantageous for the composition to also comprise a transepidermal delivery agent. Examples of transepidermal delivery agents are described in US patent publication no. 2009/005320, which is hereby incorporated by reference herein in its entirety. A suitable transepidermal delivery agent is capable of delivering the optical clearing agent intact through the epidermis into the dermis, while retaining the barrier function to prevent transepidermal water loss and xerosis. The transepidermal deliver agent may comprise two or more transepidermal penetrants working synergistically. The two or more penetrants may act through distinct biochemical pathways. In some example embodiments, the transepidermal penetrants may comprise a benzyl alcohol and a lecithin organogel. For some embodiments where a transepidermal delivery agent is incorporated into the composition, irradiation may be performed within a few minutes of topical application of the composition.

In an example embodiment, the composition comprises a combined lipophilic PPG-based polymer/hydrophilic PEG-based polymer optical clearing agent at a 50% concentration (w/w) with a transepidermal delivery agent comprising 2% benzyl alcohol (w/w) and 0.6% lecithin organogel (w/w) in a cosmetic emulsion. The inventor has determined that a transepidermal delivery agent comprising 2% benzyl alcohol (w/w) and 0.6% lecithin organogel (w/w) resulted in penetration of a compound with a minimal concentration of 0.25 g/mL through the epidermal layer through to the dermal layer within 30 minutes of application to the skin surface.

The composition may be provided in blister packaging containing for example 1 mL to 50 mL. The blister packaging may be provided with an absorbent applicator for massaging the composition onto the subject's skin overlying the target area. The massaging action partially disrupts the barrier function of the stratum corneum while the composition is being applied, sustaining a breach of the barrier function while simultaneously delivering the optical clearing agent to enhance the penetration of light. The combination blister packaging/absorbent applicator may for example be the SNAPPLICATOR™ product made by Tapemark, West St. Paul, Minn.

(iii) Enhanced Lipomodulation

In some embodiments of the invention, irradiation is followed by application of a composition to the skin overlying the target adipose tissue for lipomodulation, i.e., stimulation of lipolysis and/or inhibition of lipogenesis. The composition comprises one or more lipomodulation agents.

According to some embodiments, the lipomodulation agents may comprise:
A phosphodiesterase inhibitor to stimulate lipolysis. Phosphodiesterase inhibitors induce cyclic AMP accumulation in the adipocyte cellular membranes (by means of adenylate cyclase formation) and, thereby, increases triglyceride lipase levels, which results in hydrolysis of cellular triglycerides into fatty acids and glycerol allowing them to be absorbed and removed via the bloodstream.
A circulating lipoprotein lipase (LPL) inhibitor to inhibit lipogenesis. LPL hydrolyzes circulating lipoprotein triglycerides, resulting in triglyceride storage within adipocytes.
A nitric oxide (NO) secretion stimulator to stimulate lipolysis. NO acts as an endogenous messenger to the adipocyte cell membrane receptor, activating release of fatty acids and glycerol by the adipocytes.
An antioxidant activity enhancer to reduce reactive oxygen species (ROS) to inhibit lipogenesis. ROSs inhibit lipolysis and stimulate lipogenesis.

According to example embodiments, the composition may comprise:
an active ingredient combination of theophylline acetic acid, alginic acid and methylsilanetriol;
an active ingredient combination of caffeine, mannuronic acid and methylsilanetriol;
an active ingredient combination of L-arginine and methylsilanetriol; or
an active ingredient combination of L-arginine, caffeine, and methylsilanetriol

In some embodiments, the composition may comprise two or more lipomodulation agents.

In some embodiments, the composition may also include agents known to enhance biosynthesis of the extracellular matrix (ECM). Enhancing ECM biosynthesis may reverse predisposing factors responsible for localized lipodystrophy.

In some embodiments, the composition may further comprise a transepidermal delivery agent such as those described in US patent publication no. 2009/005320. The transepidermal delivery agent may comprise two or more transepidermal penetrants working synergistically. The two or more penetrants may act through distinct biochemical pathways. In some example embodiments, the transepidermal penetrants may comprise a benzyl alcohol and a lecithin organogel.

A composition for lipomodulation according to an example embodiment comprises the following ingredients:
1. Methylsilanol Carboxymethyl Theophylline Alginate (6% w/w). This ingredient induces cyclicAMP synthesis, aids in in connective tissue and microcirculation regeneration, has anti-inflammatory activity (to reduce edema), and inhibits lipoprotein lipase (LPL).
2. Silanetriol Arginate (5% w/w). This ingredient stimulates the secretion of nitric oxide (NO) resulting in the release of fatty acid and glycerol (lipolysis).
3. L-Ergothioneine (1.0% w/w). This ingredient has high anti-oxidant activity and thus complements lipolysis.
4. Stearyl Glycyrrhetinate (0.1% w/w in 2% hydrogenated lecithin). This ingredient reduces inflammation. The hydrogenated lecithin enhances penetration.
5. Dipropylene Clycol (1% w/w)+Palmaria Palmata (5% w/w). This ingredient inhibits adipocyte differentiation, increases collagen synthesis, and increases microcirculation.
6. Chlorella Vulgaris Extract (0.5% w/w). This ingredient promotes TIMP-1 and TIMP-3, promotes collagen and elastin synthesis by supplying essential amino acids, and inhibits matrix metalloproteinases (MMPs).
7. Hydolyzed Lupin Protein (0.5% w/w). This ingredient blocks activity of all three UV-induced MMPs, collagenase, stromylysin-1 and gelatinase B.
8. Ascorbyl Tetraisopalmitate (0.5% w/w). This ingredient is a powerful anti-oxidant and precursor of collagen.
9. Glucosamine HCI, Algae Extract, Yeast Extract, Urea (3% w/w). This ingredient increases skin firmness by promoting collagen synthesis.
10. Caprylic/Capric Triglyceride, Hydrogenated Vegetable Oil, Polygonum Fagopyrum Seed Extract (2% w/w). This ingredient contains two phytosterol inhibitors, and reduces lipogenesis.
11. Butylene Glycol, Theobroma Cacao Extract (2% w/w). This ingredient inhibits phosphodiesterase III.

In some embodiments, the compositions may comprise one or more of the above-listed ingredients in similar or different concentrations.

In some embodiments, the compositions are topically applied prior to irradiation. For example, the composition may be applied to one or more targeted portions of a subject's body, and following sufficient time for transdermal transportation of the active ingredient(s) one or more LED or laser applicators can be placed in contact with, or secured to, the one or more targeted portions for application of irradiation. Each applicator may include a plurality of LEDs or laser diodes.

In some embodiments, the compositions may be applied as a morning formulation and an evening formulation, with irradiation treatment after each application. In some embodiments the morning formulation may have one or more different active ingredients compared to the evening formulation.

Clinical studies applying compositions of the present invention twice daily for 60 days to two groups of 25 subjects were performed. The compositions comprised active ingredients 1-11 listed above in combination with a transepidermal delivery agent comprising a benzyl alcohol and a lecithin organogel.

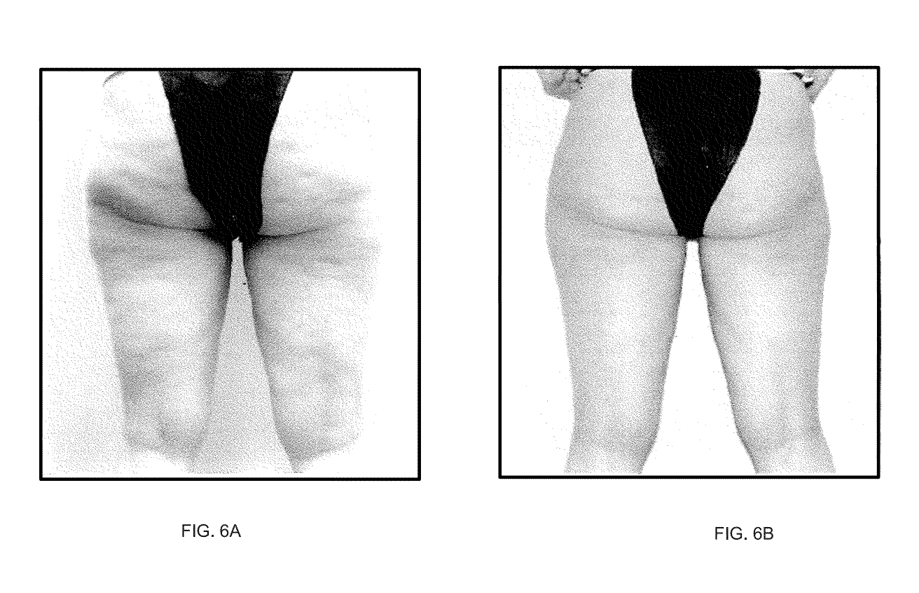

One study utilized a morning cream formulation and an evening cream formulation, each comprising active ingredients 1-11 listed above to treat cellulite. The cream formulations were evenly applied over an area of the subject's thighs with clinical manifestations of irregular skin contours or dimpling of the skin. The subjects all reported evidence of clinical improvement. Comparison of the pre-treatment with the post-treatment photos demonstrated clinical improvement in each case. FIGS. 6A and 6B are respectively pre-treatment and post-treatment photos of the thighs of a representative subject.

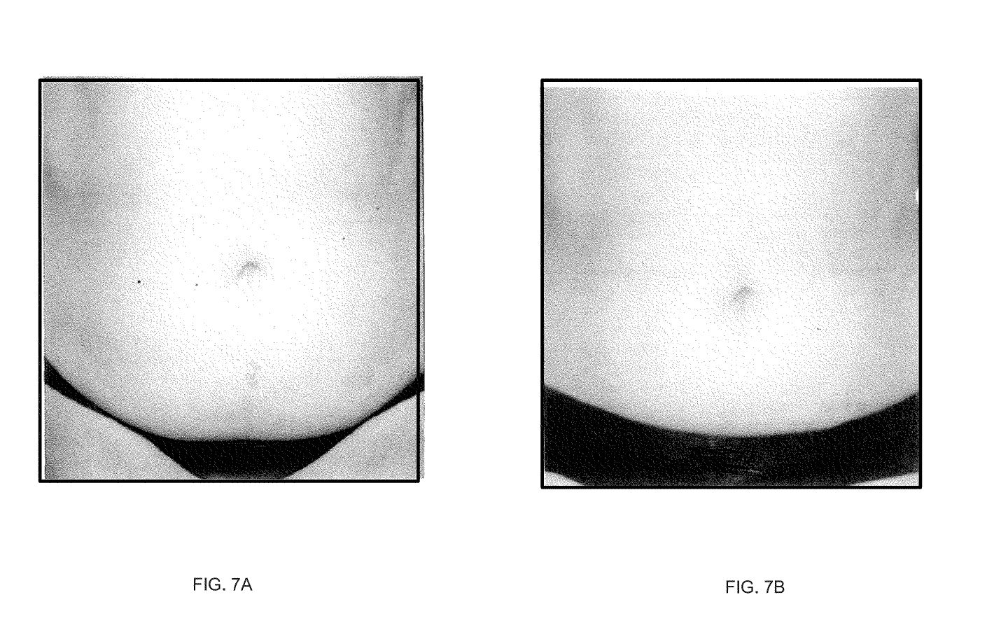

The second study utilized 25 subjects with clinical manifestations of cellulite followed for 60 days. Each subject applied a formulation comprising the composition twice daily. Every subject noticed clinical improvement at 30 days with improvement out to completion of the follow-up. FIGS. 7A and 7B are respectively pre-treatment and post-treatment photos of the abdomen of a representative subject.

### (iv) Skin Tone Restoration

The inventor has determined that light based lipolysis of adipose tissue can result in a slackening of the skin overlying the treated adipose tissue. This slackening is believed to be due to liquefaction of fats within the adipose cells, movement of the liquefied fat from the disrupted adipose cells into interstitial spaces, and resulting destabilization in the dermal-epidermal junction.

According to another embodiment of the present invention, a skin tone restoration agent may be applied to affected skin surfaces to restore skin tone after irradiation. Skin tone may be restored by promoting collagen and proteoglycan synthesis and/or promoting collagen cross-linking. The skin tone restoration agent comprise one or more of:
an antioxidant such as α-lipoic acid or ascorbic acid;
a metallic catalyst such as copper;
an essential amino acid such as methionine and/or cysteine; and
a bioflavonoid such as proanthocyanidin.

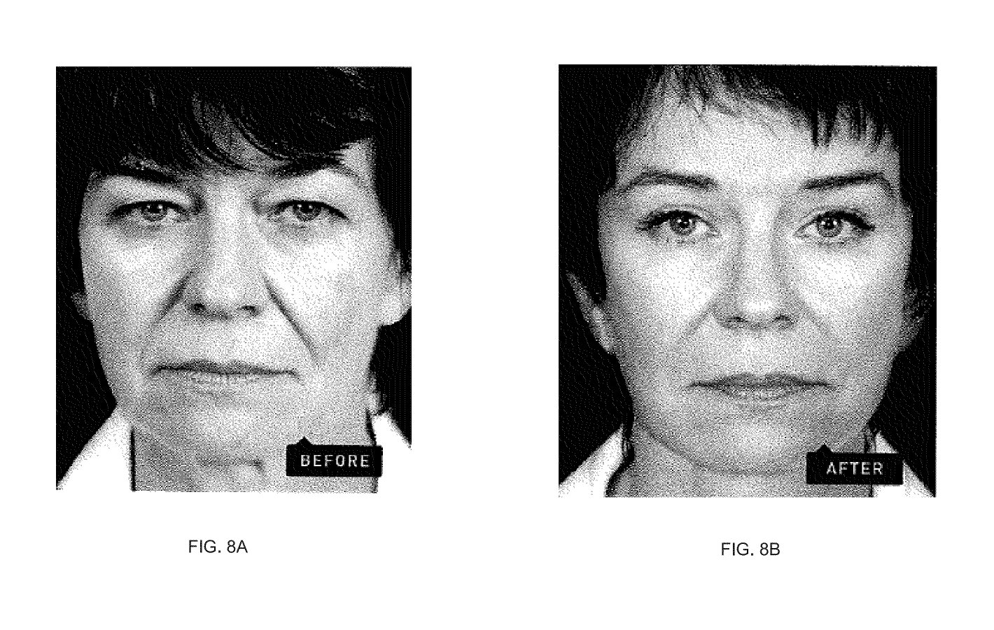

The inventor has determined that it would be advantageous to combine the skin tone restoration agent with a transepidermal delivery agent such as those described in US patent publication no. 2009/005320. The transepidermal deliver agent may comprise two or more transepidermal penetrants working synergistically. The two or more penetrants may act through distinct biochemical pathways. In some example embodiments, the transepidermal penetrants may comprise a benzyl alcohol and a lecithin organogel. FIGS. 8A and 8B are respectively pre-treatment and post-treatment photos showing visible improvement in skin tone of the face of a subject after irradiation treatment patients, where treatment involved application of a skin tone restoration composition comprising 1.0% (w/w) proanthocyandin and a transepidermal delivery agent comprising a benzyl alcohol and a lecithin organogel.

Although the present invention has been described with reference to certain exemplary embodiments thereof, in view of numerous changes and variations that will be apparent to persons skilled in the art, the scope of the present invention is to be considered limited solely by the appended claims.

1-15. (canceled)

16. A method for inducing lipolysis in a target adipose tissue site, the method comprising:
(a) identifying a target adipose tissue site in a patient in need of lipolysis;
(b) providing a light applicator comprising a plurality of LEDs arranged on a substrate; and
(c) irradiating a skin surface overlying the target adipose tissue site with non-collimated, overlapping LED light at a wavelength ranging from 625 nm to 680 nm from the light applicator.

17. A method according to claim 16 wherein spacing between adjacent LEDs on the substrate is no greater than 1.0 cm.

18. A method according to claim 17 wherein spacing between adjacent LEDs on the substrate is no greater than 0.5 cm.

19. A method according to claim 16 wherein the substrate comprises a deformable material.

20. A method according to claim 16 wherein the light applicator comprises a cover for covering at least the plurality of LEDs, wherein the cover is transparent to light emitted by the plurality of LEDs.

21. A method according to claim 20, wherein the cover comprises a flat surface for contacting the skin surface of the patient, and irradiating the skin surface comprises contacting the skin surface of the patient with the light applicator.

22. A method according to claim 21, wherein the cover comprises a deformable material.

23. A method according to claim 21, wherein irradiating the skin surface comprises conforming the light applicator to a contour of the skin surface of the patient.

24. A method according to claim 16, wherein step (a) comprises identifying a plurality of target adipose tissue sites in need of lipolysis, step (b) comprises providing a plurality of light applicators each comprising a plurality of LEDs arranged on a substrate, and step (c) comprises irradiating skin surfaces overlying the target adipose tissue sites with non-collimated, overlapping LED light at a wavelength ranging from 625 nm to 680 nm from the plurality of light applicators.

25. A method according to claim 24 comprising controlling the plurality of light applicators with a control device.
